(19) United States
(12) Patent Application Publication
(10) Pub. No.: US 2017/0206318 A1
(43) Pub. Date: Jul. 20, 2017

(54) MEDICAL SYSTEM AND MEDICAL DEVICE

(71) Applicant: OLYMPUS CORPORATION, Tokyo (JP)

(72) Inventor: Takashi OZAKI, Tokyo (JP)

(73) Assignee: OLYMPUS CORPORATION, Tokyo (JP)

(21) Appl. No.: 15/476,041

(22) Filed: Mar. 31, 2017

Related U.S. Application Data
(63) Continuation of application No. PCT/JP2016/060191, filed on Mar. 29, 2016.

(30) Foreign Application Priority Data
Apr. 13, 2015 (JP) 2015-081745

(51) Int. Cl. G06F 19/00; A61B 1/05; H04L 29/08; A61B 1/00

(52) U.S. Cl. CPC G06F 19/321; A61B 1/00009; A61B 1/05; A61B 1/00011; H04L 67/06; H04L 67/12

(57) ABSTRACT

A medical device includes: a communication data file generator that generates a data file that is transceivable by using a prescribed protocol that is different from a communication protocol of a DICOM standard, the data file including metadata based on medical information relating to a subject; and a transmitter that outputs the data file generated by the communication data file generator to a network drive that does not implement an application relating to DICOM, the network drive being connected via a network specified by the prescribed protocol.

### CROSS-REFERENCE TO RELATED APPLICATIONS

This application is based upon and claims the benefit of priority of the prior Japanese Patent Application No. 2015-081745, filed Apr. 13, 2015, the entire contents of which are incorporated herein by reference.

This is a Continuation Application of PCT Application No. PCT/JP2016/060191, filed Mar. 29, 2016, which was not published under PCT Article 21(2) in English.

### FIELD

The present invention relates to a medical system including a medical device that records medical information, and in particular, a medical system that performs communication between a medical device and an external device via a network, and stores medical information in the external device.

### BACKGROUND

At the medical site, it is necessary to perform examination, photograph a subject by using a medical device such as an endoscope device so as to obtain data such as a medical image, refer to the obtained data such as a medical image after the examination, and perform diagnosis or generate a report on an examination result. Therefore, medical systems have been widely used that each include a medical device, and an external device, such as a server device, that stores data such as a medical image, the medical device and the external device being mutually connected via a network. A user such as a doctor accesses the external device after the examination, refers to the data such as a medical image, and, for example, performs diagnosis or generates a report.

Here, the DICOM standard specifies a medical image such as an endoscopic image, and a telecommunications standard of the medical image. In a medical system that conforms to the DICOM standard, image data obtained by a medical device in examination is converted into data in the DICOM format. The obtained data in the DICOM format is transferred to an external device, such as a server, that is connected via a network, and the data is stored in a transfer destination device (for example, Japanese Laid-Open Patent Publication No. 2010-128659 or Japanese Laid-Open Patent Publication No. 2008-036353).

As a known technology, a technology for performing communication between a device that conforms to the DICOM standard and a medical device that does not conform to the DICOM standard via a network for DICOM communication has also been disclosed (for example, Japanese Laid-Open Patent Publication No. 2003-248723).

In addition, a technology has been disclosed in which an image file is transceived between a medical device and a server by using a general-purpose protocol in the case of a configuration in which neither the medical device nor the server conforms to the DICOM standard (for example, Japanese Laid-Open Patent Publication No. 2009-213790).

As described above, not all of the devices that configure a medical system conform to the DICOM standard, and some devices that do not conform to the DICOM standard may be used. In addition, as an example, a large-capacity file server such as a network attached storage (NAS) is desirable for a destination to store data such as a medical image, but application software needed to perform DICOM communication is not installed in the large-capacity file server.

In consideration of convenience of a user, or the like, it is desirable that data obtained by a medical device in examination be stored in the DICOM format in an external device such as a NAS, which is a large-capacity storage, such that the user can use the data later.

### SUMMARY

A medical system in an aspect cf the present invention includes: a medical device that includes a storage unit that stores medical information relating to a subject, a communication data file generator that generates a data file that is transceivable by using a prescribed protocol that is different from a communication protocol of a digital imaging and communication in medicine (DICOM) standard, the data file including metadata based on the medical information, and a transmitter that transmits the data file generated by the communication data file generator to an outside that is connected via a network specified by the prescribed protocol; and a network drive that is configured to be able to record the data file that is output from the transmitter, is connected to the medical device via the network specified by the prescribed protocol, and does not implement an application relating to DICOM.

A medical device in another aspect of the present invention includes: a storage unit that stores medical information relating to a subject; a communication data file generator that generates a data file that is transceivable by using a prescribed protocol that is different from a communication protocol of a digital imaging and communication in medicine (DICOM) standard, the data file including metadata based on the medical information; and a transmitter that outputs the data file generated by the communication data file generator to a network drive that does not implement an application relating to DICOM, the network drive being connected via a network specified by the prescribed protocol.

### BRIEF DESCRIPTION OF DRAWINGS

FIG. 1 is a block diagram illustrating a medical system according to an embodiment.

FIG. 2 illustrates a method for transferring examination data in a medical system according to an embodiment.

FIG. 3A illustrates a conventional method for transferring examination data in a medical system (no. 1).

FIG. 3B illustrates a conventional method for transferring examination data in a medical system (no. 2).

FIG. 4 illustrates an example of the structure of metadata that is added when a medical system according to an embodiment generates a data file.

FIG. 5A a diagram explaining an effect at the time of accessing a network drive and using stored data such as an image.

FIG. 5B is a diagram explaining a conventional technology in comparison with FIG. 5A.

FIG. 6A is a diagram explaining another effect at the time of accessing a network drive and using stored data such as an image.

FIG. 6B is a diagram explaining a conventional technology in comparison with FIG. 6A.

### DESCRIPTION OF EMBODIMENTS

An embodiment of the present invention is described in detail with reference to the drawings.

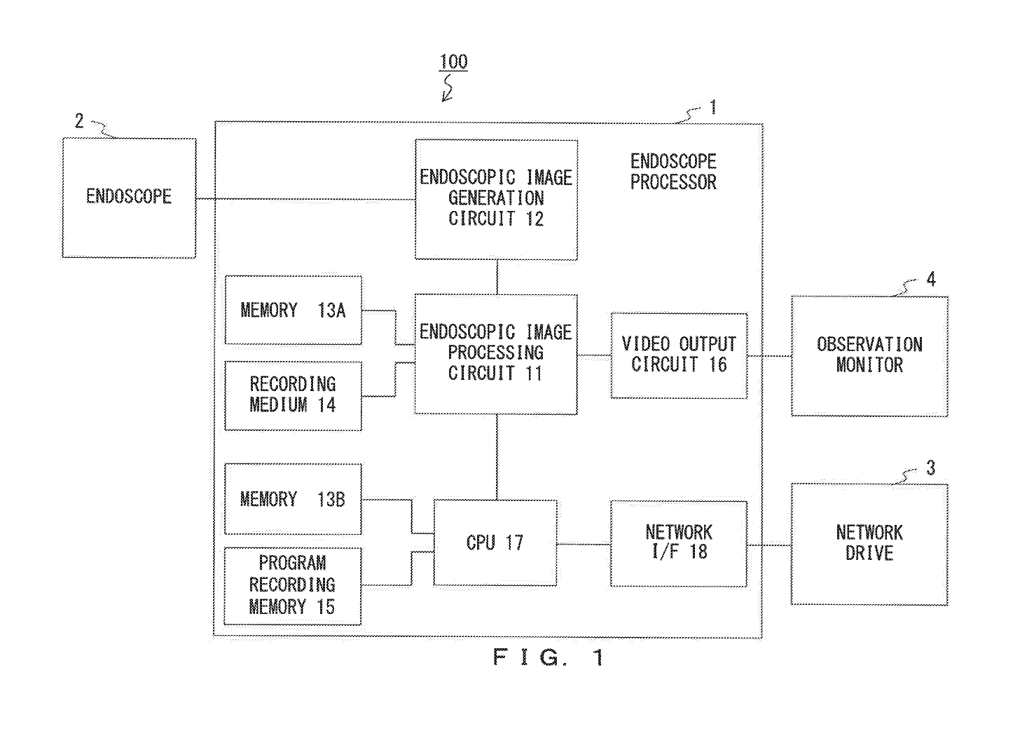

FIG. 1 is a block diagram illustrating a medical system according to an embodiment. A medical system 100 of FIG. 1 includes an endoscope (hereinafter simply referred to as a scope) 2, an endoscope processor (hereinafter simply referred to as a processor) 1, an observation monitor (hereinafter simply referred to as a monitor) 4, and a network drive 3.

The scope 2 can be inserted into a body cavity of a subject, and the scope 2 captures an image by using an imaging unit at a distal end, and transmits an obtained image signal to the processor 1.

The processor 1 includes an endoscopic image generation circuit 12, an endoscopic image processing circuit 11, a memory 13A, a recording medium 14, a video output circuit 16, a CPU 17, a memory 13B, a program recording memory 15, and a network interface (a network I/F) 18. FIG. 1 illustrates only a configuration relating to this embodiment.

Among these components, the endoscopic image generation circuit 12 performs necessary processing on the image signal that is input from the scope 2, and generates an endoscopic image. The endoscopic image processing circuit 11 processes an image signal that is sequentially input from the scope 2 so as to generate an endoscopic image, and outputs the endoscopic image to the video output circuit 16. The video output circuit 16 generates an image obtained by combining the endoscopic image input from the endoscopic image processing circuit 11, for example, with information such as characters, and outputs the generated image to the monitor 4. The monitor 4 displays the image received from the video output circuit 16 as a live video. The endoscopic image processing circuit 11 uses the memory 13A, for example, to temporarily store a generated frame image at the time of performing image processing.

In addition, when the CPU 17 recognizes an instruction from a user, such as an operation of an operator to depress a release button, the endoscopic image processing circuit 11 fetches a prescribed frame image in accordance with a command from the CPU 17. The endoscopic image processing circuit 11 records the fetched image as a still image in the recording medium 14. An image file generated in the DICOM format is recorded in the recording medium 14. The CPU 17 reads the recorded still image from the recording medium 14 in accordance with an instruction from a user, for example, after examination, and outputs the still image to the network drive 3 via the network interface 18.

The network interface 18 transfers the image to the network drive 3 by using a general-purpose communication protocol, not a communication protocol specified by the DICOM standard. Examples of the general-purpose communication protocol include the file transfer protocol (FTP), the server message block/common internal file system (SMB/CIFS), the network file system (NFS), and the apple filing protocol (AFP).

The program recording memory 15 stores a program for controlling a process for transferring examination data, such as image data, that is obtained in endoscopy to the network drive 3. The CPU 17 realizes a method for transferring examination data according to this embodiment by reading this control program from the program recording memory 15 and expanding the control program in the memory 13B and executing the control program.

When the CPU 17 recognizes an instruction to output a still image to the network drive 3, the CPU 17 controls the endoscopic image processing circuit 11 and the network interface 18 so as to read examination data including image data from the recording medium 14. The examination data read from the recording medium 14 is configured by data in the DICOM format, and the examination data includes various data relating to examination, such as an examination date and time or a patient, in addition to data of an endoscopic image. The CPU 17 generates a data file from the examination data, and transfers the generated data file to an external device (in the example of FIG. 1, the network drive 3) via the network interface 18 by using a general-purpose communication protocol.

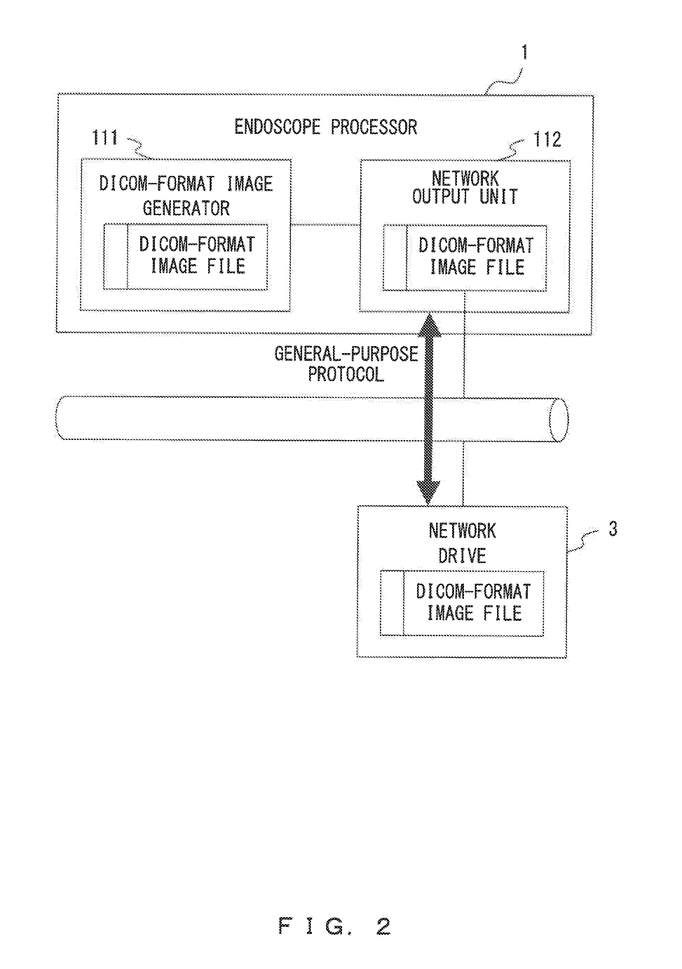

A method in which the medical system according to this embodiment transfers the data file generated from the examination data from the processor 1 is described below in detail with reference to FIG. 2 and the like.

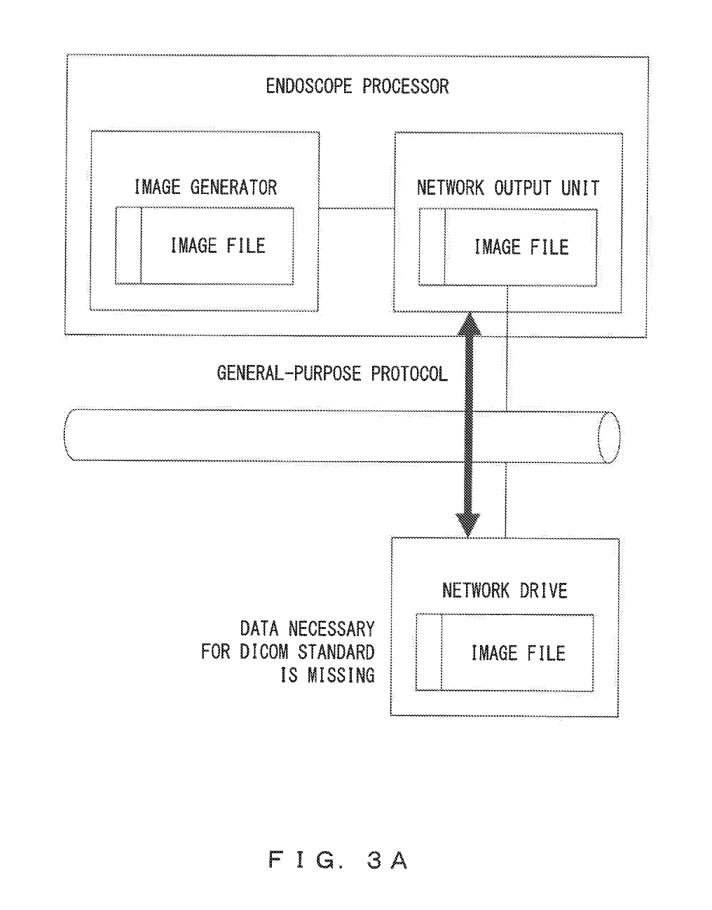

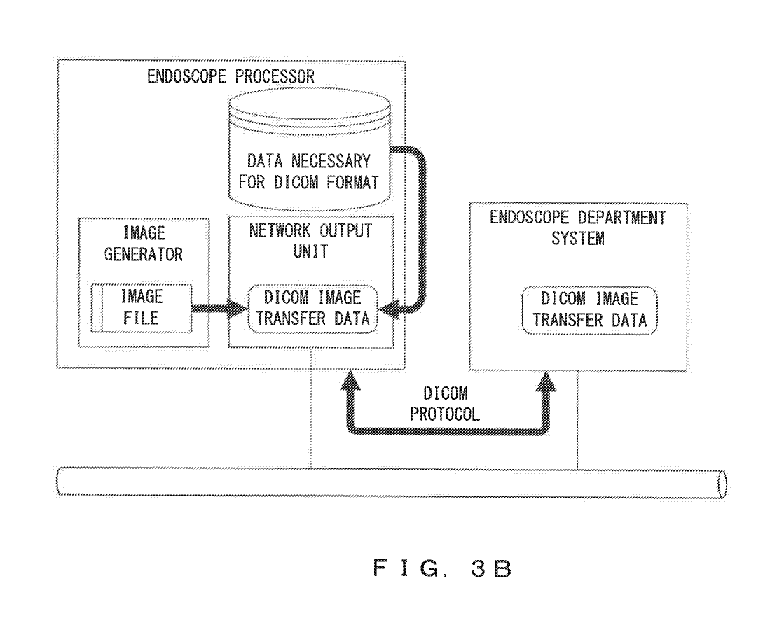

FIG. 2 illustrates a method for transferring examination data in the medical system 100 according to this embodiment. FIGS. 3A and 3B illustrate conventional methods for transferring examination data in a medical system.

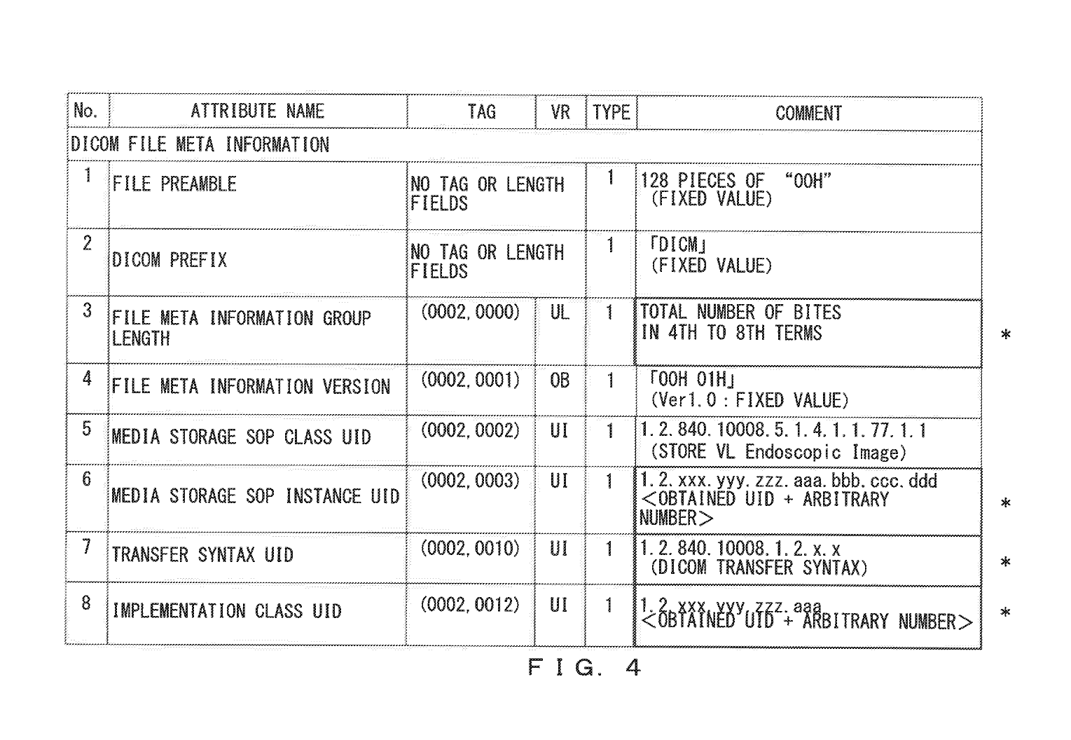

In the medical system 100 according to this embodiment, a DICOM-format image generator 111 of the processor 1 first generates a data file in the DICOM format from examination data including data of a still image in the DICOM format that is recorded in the recording medium 14. This corresponds to an operation in which the CPU 17 of FIG. 1 instructs the endoscopic image processing circuit 11 to read examination data such as data of a still image from the recording medium 14, and generates a data file from the read data. The data file can be transceived by using a general-purpose communication protocol. A method for generating a data file will be described later in detail with reference to FIG. 4.

Then, a network output unit 112 of the processor 1 transfers the data file in the DICOM format that has been generated by the DICOM-format image generator 111, to the network drive 3 by using a general-purpose communication protocol such as the FTP. This corresponds to an operation in which the CPU 17 of FIG. 1 transmits a generated file to the network drive 3 via the network interface 18.

Conventionally, there is a method in which a processor generates a data file in a format other than the DICOM format, and transfers and stores the data file in a network drive that does not conform to the DICOM standard, such as a NAS, as illustrated in FIG. 3A. In this case, a communication protocol specified by the DICOM cannot be used, and a file is transferred by using a general-purpose communication protocol.

In a case in which data in the DICOM format is managed, the configuration illustrated in FIG. 3B is employed. In the configuration illustrated in FIG. 3B, in transferring a data file including, for example, image data that is generated and stored by a processor to an external server (in FIG. 3B, this is provided in an endoscope department system), data is converted into DICOM-format data, and the DICOM-format data is transferred. The “DICOM-format data” refers to a data set according to the “VL Endoscopic Image Storage”, which is an example of an SOP class defined in the “DICOM standard PS3.3”. In this case, the data file is encoded in a method specified in the DICOM standard PS3.5 and the data set (the encoded data file) is transmitted in a method specified in the DICOM standard PS3.7 to an endoscope department system including a server device that stores data. A communication protocol used in this case is a communication protocol specified by the DICOM.

In the conventional method illustrated in FIG. 3A, an image file can be transferred and stored in a large-capacity storage such as a NAS, but information necessary for the DICOM standard is missing from data stored in a network drive. The data stored in the network drive is used to generate a report or perform diagnosis. When prescribed information is missing from the data stored in the network drive, it cannot be said that convenience is sufficient.

In addition, in the conventional method illustrated in FIG. 3B, an output destination of an image file always needs to be a device that conforms to the DICOM standard. Accordingly, a certain cost is required for capital investment. In addition, a prescribed data input needs to be performed to generate data to be transferred in the DICOM format in the processor 1.

On the other hand, in the method illustrated in FIG. 2 for transferring examination data according to this embodiment, the processor 1 generates a file including all necessary information specified by the DICOM standard, and transfers the generated file to the network drive 3 by using a general-purpose communication protocol. Because the general-purpose communication protocol is used, a file can be transferred regardless of whether a device that is a transfer destination of the file conforms to the DICOM standard. In addition, because a data file stored in the network drive 3 is generated in the DICOM format, the network drive 3 does not always need to implement an application of a DICOM service (a function provided according to the DICOM standard). Accordingly, a large-capacity storage such as a NAS can be used as a storage destination of a data file.

When the entirety of a medical system that is configured by a plurality of devices such as a medical device or a server needs to conform to the DICOM standard, a large burden is imposed on a medical institution. However, according to this embodiment, a large-capacity storage, such as a NAS, that does not conform to the DICOM standard can be used as a storage destination of examination data, and this results in the suppression of an initial investment cost. Further, the suppression of the initial investment cost results in the spread of the medical system 100 that conforms to the DICOM standard.

According to this embodiment, a data file is generated by adding metadata specified in the DICOM standard PS3.10 to the above “VL Endoscopic Image Storage”, which is an example of an SOP class of the DICOM. This is described below with reference to FIG. 4.

FIG. 4 illustrates an example of the structure of metadata. that is added when the medical system 100 according to this embodiment generates a data file.

The DICOM-format image generator 111 of the processor 1 adds the metadata “DICOM File Meta Information” of FIG. 4 at the top of data defined in the above SOP class “VL Endoscopic Image Storage” so as to generate a data file. The DICOM-format image generator 111 transmits the generated data file to the outside.

According to the DICOM standard PS3.10, the metadata of FIG. 4 is used to record data such as a medical image in a medium such as a compact disc (CD). Data is encapsulated by adding the metadata of FIG. 4 to the data such as a medical image.

According to this embodiment, a data file is generated by adding metadata that is specified for the purpose of encapsulating image data in order to output the image data to a local storage such as a CD-ROM. Consequently, an image file in the DICOM format can be stored in the network drive 3 that does not conform to the DICOM standard, such as a NAS. Hereinafter, adding the metadata of FIG. 4 to the above data of the SOP class is referred to as “filing” data.

In the metadata of FIG. 4, items marked “*” are essential input items. A device that accesses the network drive 3 refers to the metadata of FIG. 4 such that the device can recognize that a data file stored in the network drive 3 is a file including DICOM image data, and that the device can identify each of the data files. The CPU 17 of the processor 1 can set a required value in each of the items in the metadata of FIG. 4 by using, for example, information at the time when the endoscopic image processing circuit 11 records data of a still image in the recording medium 14.

When a user refers to an image stored in the network drive 3 in order to generate a report or perform diagnosis, a data file in the network drive 3 needs to be transferred to another device or system that the user uses via a network. Filing image data or the like by using the method above also exhibits an effect at the time of accessing image data or the like that is stored in the network drive 3 from another device or the like. This is described below with reference to the drawings.

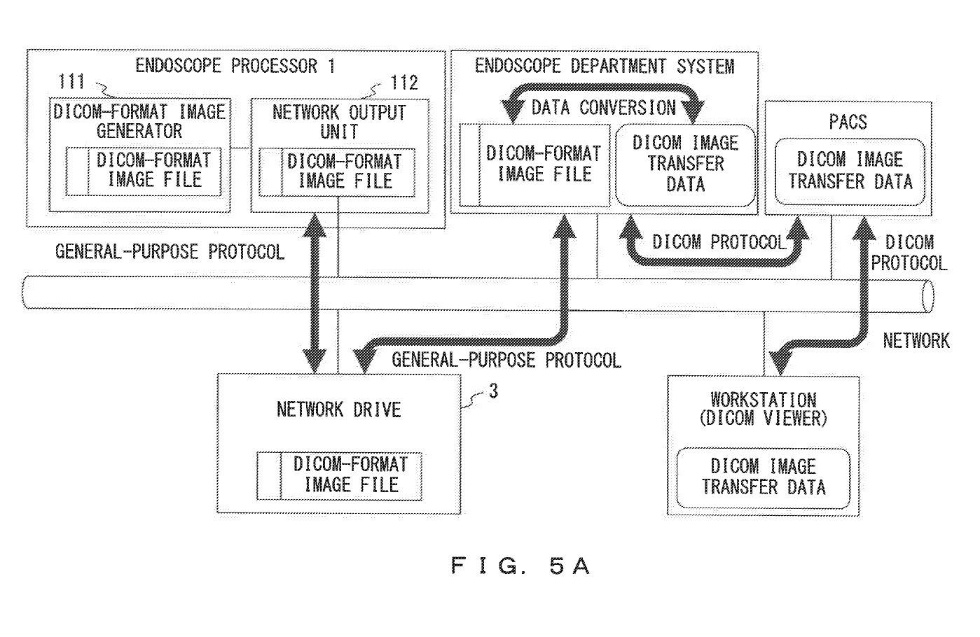

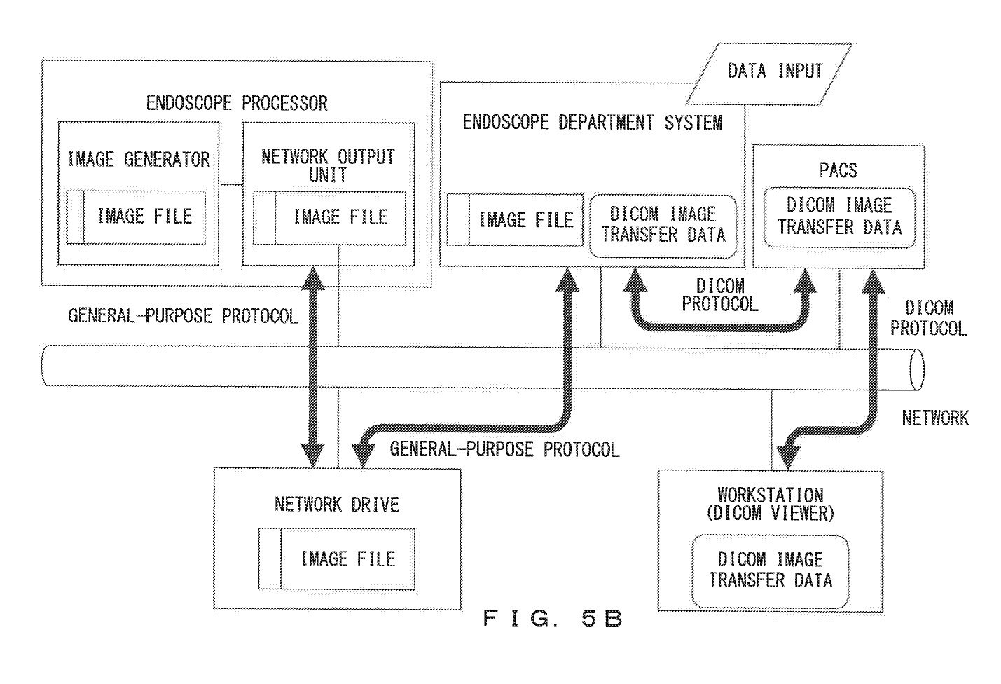

FIG. 5A is a diagram explaining an effect at the time of accessing a network drive 3 and using stored data such as an image. FIG. 5B is a diagram explaining a conventional technology in comparison with FIG. 5A.

The endoscope department systems of FIGS. 5A and 5B are systems that are provided in order for the processor 1 to manage image data stored in an external device (in this example, the network drive 3). A user accesses a management system, picture archiving and communication systems (PACS), via a viewer (a work station), and refers to data, such as an endoscopic image, via the PACS.

In the exemplary configurations illustrated in FIGS. 5A and 5B, it is assumed that a system and a device (the endoscope department system, the PACS, and the work station) that access examination data including an endoscopic image that is obtained by the processor 1 conform to the DICOM standard. It is also assumed that the network drive 3 is configured by a NAS that does not conform to the DICOM standard.

As described above, a data file with the metadata of FIG. 4 added has been stored in the network drive 3 by using the method according to this embodiment for transferring examination data from the processor 1 to the network drive 3.

The endoscope department system performs communication with the network drive 3 by using a general-purpose protocol such as the FTP, converts a received data file, and obtains data to be transferred (DICOM transfer data). The DICOM transfer data is obtained by deleting the metadata of FIG. 4 from a data file obtained by the above filing process. The DICOM transfer data after conversion is provided to the PACS and the work station that are mutually connected to the endoscope department system via a network. The endoscope department system, the PACS, and the work station transceive the DICOM transfer data obtained by data conversion by using a communication protocol specified by the DICOM.

As described above, the endoscope department system can obtain the DICOM transfer data by performing only a process for deleting metadata added in a prescribed position from a data file received from the network drive 3. Stated another way, the endoscope department system does not need to perform, for example, an operation to input additional data in order to obtain the DICOM transfer data. Even in a case in which a device that does not conform to the DICOM standard, such as a NAS, is employed in the network drive 3, a user's operation is not needed at the time of accessing the network drive 3 and using data such as an endoscopic image, and this results in improvements in work efficiency.

Also in the conventional configuration illustrated in FIG. 5B, in using a data file including image data in a NAS, the endoscope department system needs to perform conversion into DICOM-format data. However, in the conventional configuration, DICOM transfer data needs to be obtained from a file that does not include all necessary information for the DICOM standard. Accordingly, a user needs to perform an operation to add and input data necessary for data conversion via the endoscope department system and the like.

A configuration in which data necessary for the DICOM standard is stored in a NAS is also considered. By employing the configuration above, the endoscope department system can receive the data necessary for the DICOM standard, together with a data file including image data, from the NAS, and can perform data conversion according to the data, and therefore the endoscope department system does not need to input data. However, in the case of the configuration above, the endoscope department system that receives data from the NAS needs to implement dedicated application software for generating DICOM image transfer data.

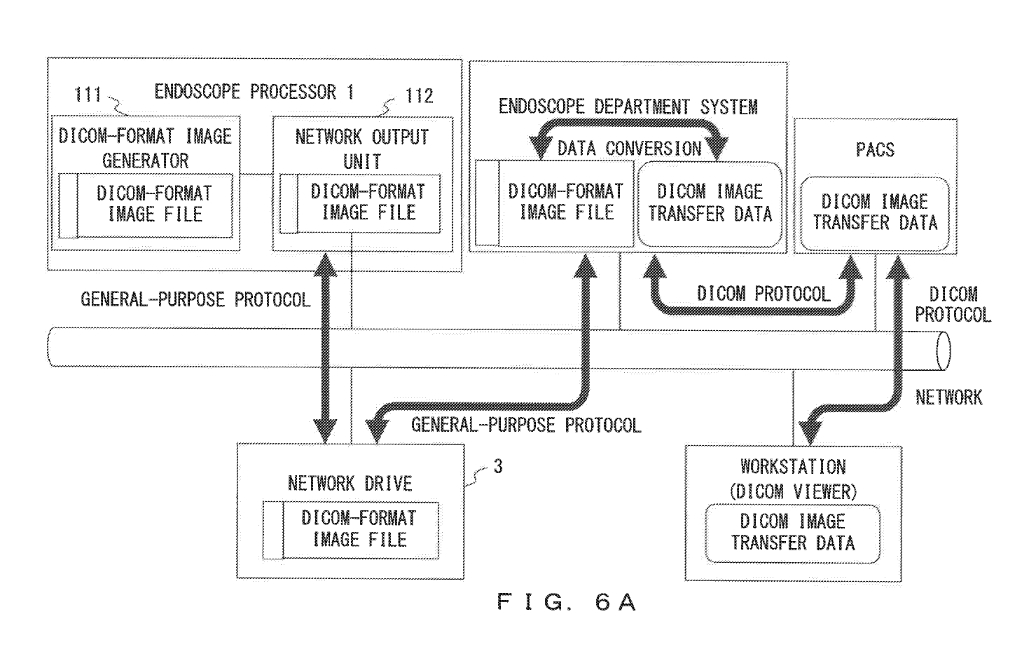

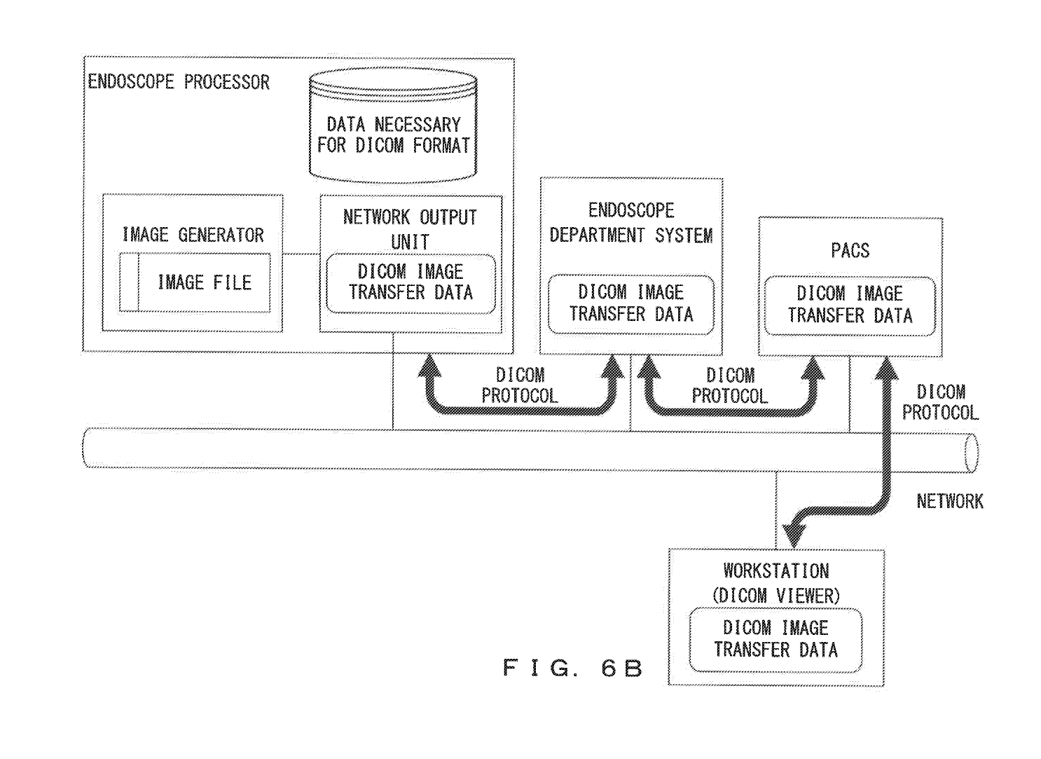

FIG. 6A is a diagram explaining another effect at the time of accessing a network drive 3 and using stored data such as an image. FIG. 6B is a diagram explaining a conventional technology in comparison with FIG. 6A.

Also in the configuration of FIG. 6A, similarly to FIGS. 5A and 5B, an endoscope department system that manages image data that a processor 1 stores in the network drive 3, a PACS, and a work station that is a viewer are connected via a network. The endoscope department system, the PACS, and the work station conform to the DICOM standard, and the network drive 3 is configured by a NAS that does not conform to the DICOM standard. On the other hand, in the configuration illustrated in FIG. 6B, it is assumed that data such as an image is stored in a server device of an endoscope department system, and that each system or device conforms to the DICOM standard.

The endoscope department system that manages examination data including an endoscopic image may need to restart the system as a result of software updating or maintenance. In the configuration illustrated in FIG. 6A, even when the endoscope department system goes down, image data has been stored in the NAS (the network drive 3), and therefore there is no influence on the output of the image data from the processor 1 to an external device. Stated another way, the endoscope department system does not need to manage to transfer and store image data recorded in the recording medium 14 of the processor 1 in examination to/in the network drive 3 such as the NAS. Therefore, even when the endoscope department system is restarted, a situation does not occur in which image data cannot be transferred from the processor 1 to the network drive 3. Accordingly, in the endoscope department system, software updating, maintenance, or the like can be performed at any time without being limited to the time after endoscopy, holidays, or the like.

On the other hand, in the conventional configuration illustrated in FIG. 6B, the endoscope department system manages to transfer image data from the processor 1 to an external device. Accordingly, a server of the endoscope department system needs to be running and operating at all times during examination, and software updating or maintenance of the endoscope department system needs to be performed during a time period or on a day that does not affect examination or the like.

As described above, in the medical system 100 according to this embodiment, when transferring and storing examination data including endoscopic image data in an external device, the processor 1 performs filing by adding metadata to the examination data, and transmits the examination data by using a general-purpose communication protocol such as the FTP. By employing a configuration that enables data to be transferred by using the general-purpose communication protocol, a large-capacity storage that does not conform to the DICOM standard, such as a NAS, can be used as the network drive 3 that is a storage destination of data. In addition, filing has been performed on examination data to be stored in the network drive 3, and therefore when examination data is accessed from a device that conforms to the DICOM standard in order to perform diagnosis or generate a report, data conversion can be easily performed without increasing tasks of a user of the medical system 100.

According to this embodiment described above, data obtained by a medical device in examination can be transferred and stored in the DICOM format in an external device that does not conform to the DICOM standard by using a simple configuration.

The above description has been made by using, as an example, processing performed when the processor 1 transfers examination data including data of an endoscopic image to the network drive 3, but the invention is not limited to this. As an example, data obtained by another medical device or data other than examination data may be transferred. The present invention includes any process for performing filing on data obtained by various medical devices and transferring the data to the network drive 3 by using a general-purpose communication protocol.

The present invention is not limited to the embodiment above with no change, and components can be varied and embodied without departing from the gist of the embodiment above in an implementing stage. Various inventions can be made by appropriately combining a plurality of components disclosed in the embodiment above. As an example, all of the components disclosed in the embodiment may be combined appropriately. Further, components disclosed in different embodiments may be combined appropriately. It goes without saying that various variations or applications can be made without departing from the spirit of the invention.

What is claimed is:

1. A medical system comprising:
a medical device that includes:
a storage unit that stores medical information relating to a subject;
a communication data file generator that generates a data file that is transceivable by using a prescribed protocol that is different from a communication protocol of a digital imaging and communication in medicine (DICOM) standard, the data file including metadata based on the medical information; and
a transmitter that transmits the data file generated by the communication data file generator to an outside that is connected via a network specified by the prescribed protocol; and
a network drive that is configured to be able to record the data file that is output from the transmitter, is connected to the medical device via the network specified by the prescribed protocol, and does not implement an application relating to DICOM.

2. The medical system according to claim 1, wherein
the communication data file generator generates the data file by adding metadata defined by the DICOM standard to the medical information.

3. The medical system according to claim 1, further comprising:
an endoscope department system that is connected to the network drive via the network specified by the prescribed protocol, wherein
the endoscope department system converts the data file that is output from the network drive into medical information that corresponds to the communication protocol of the DICOM standard.

4. The medical system according to claim 3, wherein
the endoscope department system converts the data file into the medical information that corresponds to the communication protocol of the DICOM standard by deleting the metadata included in the data file that is output from the network drive.

5. The medical system according to claim 1, wherein
the prescribed protocol is a file transfer protocol.

6. The medical system according to claim 1, wherein
the medical device is an endoscope device.

7. A medical device comprising:
a storage unit that stores medical information relating to a subject;
a communication data file generator that generate a data file that is transceivable by using a prescribed protocol that is different from a communication protocol of a digital imaging and communication in medicine (DICOM) standard, the data file including metadata based on the medical information; and
a transmitter that outputs the data file generated by the communication data file generator to a network drive that does not implement an application relating to DICOM, the network drive being connected via a network specified by the prescribed protocol.
